(19) United States
(12) Patent Application Publication
(10) Pub. No.: US 2019/0380601 A1
(43) Pub. Date: Dec. 19, 2019

(54) FECAL INCONTINENCE ALERT DEVICE AND SYSTEM AND METHOD OF USING THE SAME

(71) Applicant: GI Bionics LLC, San Diego, CA (US)

(72) Inventors: Ghassan S. Kassab, La Jolla, CA (US); Hans Gregersen, Ma On Shan (HK)

(73) Assignee: GI Bionics LLC, San Diego, CA (US)

(21) Appl. No.: 16/442,239

(22) Filed: Jun. 14, 2019

Related U.S. Application Data
(60) Provisional application No. 62/685,186, filed on Jun. 14, 2018.

(51) Int. Cl. A61B 5/03; A61F 13/42; A61B 5/053; A61B 5/07; A61B 5/00; A61B 5/20

(52) U.S. Cl. CPC A61B 5/036; A61F 13/42; A61B 5/0538; A61B 5/6847; A61B 5/746; A61B 5/202; A61F 2013/424; A61B 5/07

(57) ABSTRACT

A device insertable into the anus of a patient for obtaining bioimpedance and/or pressure data. The data can be analyzed to treat defecation related disesases. The device may be shaped to be easily inserted and alternatively or additionally comprise a dissolvable outer shell and spongy inner portion wherein the spongy inner portion may absorb liquids and sensors disposed thereon may obtain data from the liquid. A method of use for using said devices is also described.

### PRIORITY

The present patent application is related to, and claims the priority benefit of, U.S. Provisional Patent Application Ser. No. 62/685,186, filed on Jun. 14, 2018, the contents of which are hereby incorporated by reference in their entirety into this disclosure.

### BACKGROUND

Persons with fecal incontinence may involuntarily discharge fecal contents, which would ideally be prevented, or at least be addressed in a way to allow said person to have enough time to make it to a restroom prior to defecation. Devices and systems, and methods of using the same, to alert a person prior to involuntary defecation would be well received in the marketplace.

### BRIEF SUMMARY

The present disclosure includes disclosure of devices, systems, and methods to alert a person of a potential discharge event to prevent involuntary defecation.

In one embodiment of the present invention, the embodiment comprises an outer shell configured for placement into the anus; and at least one of a bioimpedance sensor or a pressure sensor.

In another embodiment of the present invention, the at least one of the bioimpedance sensor or pressure sensor is configured to obtain conductance data/information of/from anorectal tissue as well as to obtain conductance data/information of/from fecal matter.

In an embodiment of the present invention, the device is ovular in shape. In another embodiment of the present invention, the device further comprises a first enlarged portion and a smaller portion, whereby the first enlarged portion is relatively larger than the smaller portion. In another embodiment of the present invention, as in claim 1, wherein the device further comprises a second enlarged portion whereby the second enlarged portion is relatively larger than the smaller portion.

In a system of the present invention, the system comprises a device comprising an outer shell configured for placement into the anus; and at least one of a bioimpedance sensor or a pressure sensor as in claim 1, and also comprises an external device whereby said device and said external device can communicate bidirectionally.

In an alternate embodiment of the present invention, the device comprises a bioimpedance sensor comprising a first excitation electrode, a second excitation electrode, a first detection electrode and a second detection electrode. In an embodiment of the present invention, the first detection electrode and the second detection electrode are physically positioned between the first excitation electrode and the second excitation electrode.

In an embodiment of the present invention, the device further comprises additional elements which may comprise at least one of a camera, a light source, a pH sensor, a radiopaque marker, an electromagnet marker, a mucosa attached electromagnetic stimulator, a gyroscopic sensor, a strain gauge, a chemosensor, a wireless transmitter, a wireless receiver, or a printed circuit board.

In a further embodiment of the present invention, the at least one of a bioimpedance sensor or a pressure sensor is a pressure sensor and the embodiment also comprises electrical stimulating elements positioned along a relative outside of the embodiment.

In an alternate embodiment of the present invention comprises: an external capsule encapsulating a spongy portion; and sensors configured to obtain data based on the presence of liquids in the spongy portion.

In an alternate embodiment of the present invention, the external capsule comprises a material that dissolves in the presence of a liquid, and the spongy portion comprises a material configured to absorb or receive liquids.

In a further embodiment of the present invention, the embodiment further comprises a threaded element connected to the spongy portion.

In a further embodiment of the present invention, the spongy portion comprises elements or sensors configured to obtain impedance/conductance data and pressure data thereon.

In an embodiment of a method of using a device of the present invention according to the present invention the method comprises the steps of: inserting a device configured to obtain conductance data/information of/from anorectal tissue as well as to obtain conductance data/information of/from fecal matter into an anus of a patient; collecting a first set of pressure and bioimpedance data wherein the first set of data is indicative of the non presence of feces; collecting a second set of pressure and bioimpedance data wherein the second set of data is indicative of the presence of feces; and transmitting the data to an external device.

In a further embodiment of a method of using a device of the present invention, the second set of pressure and bioimpedance data comprises a pressure reading higher than a pressure reading of the first set of pressure and bioimpedance data.

In a further embodiment of a method of using a device of the present invention the second set of pressure and bioimpedance data comprises an impedance reading lower than an impedance reading of the first set of pressure and impedance data.

In an further embodiment of a method of using a device of the present invention, said device comprises an external capsule encapsulating a spongy portion; and the method further comprises the steps of: dissolving an external capsule; absorbing environmental liquids into the spongy portion; and the step of collecting a second set of pressure and bioimpedance data is performed after the step of absorbing environmental liquids into the spongy portion.

In a further embodiment of a method of using a device of the present invention said device comprises a threaded element connected to the spongy portion; and the method further comprises the step of removing the device by pulling the threaded element.

In a further embodiment of a method of using a device of the present invention said device comprises electrical stimulating elements positioned along a relative outside of the embodiment; and further comprises the steps of activating the electrical stimulating elements when the first or second set of pressure and bioimpedance data indicates a low pressure.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a device, according to an exemplary embodiment of the present disclosure;

FIG. 2 shows a device having an enlarged portion, according to an exemplary embodiment of the present disclosure;

FIG. 3 shows a device having two enlarged portions, according to an exemplary embodiment of the present disclosure;

FIG. 4 shows a diagram of a system whereby a device and an external device can directly communicate with one another, according to an exemplary embodiment of the present disclosure;

FIG. 5 shows a diagram of a system whereby a device and an external device can indirectly communicate with one another through a network, according to an exemplary embodiment of the present disclosure;

FIG. 6 shows a component diagram of a device, according to an exemplary embodiment of the present disclosure;

FIG. 7 shows a device having a capsule portion and a spongy portion, according to an exemplary embodiment of the present disclosure; and

FIG. 8 shows a device having stimulating electrodes, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

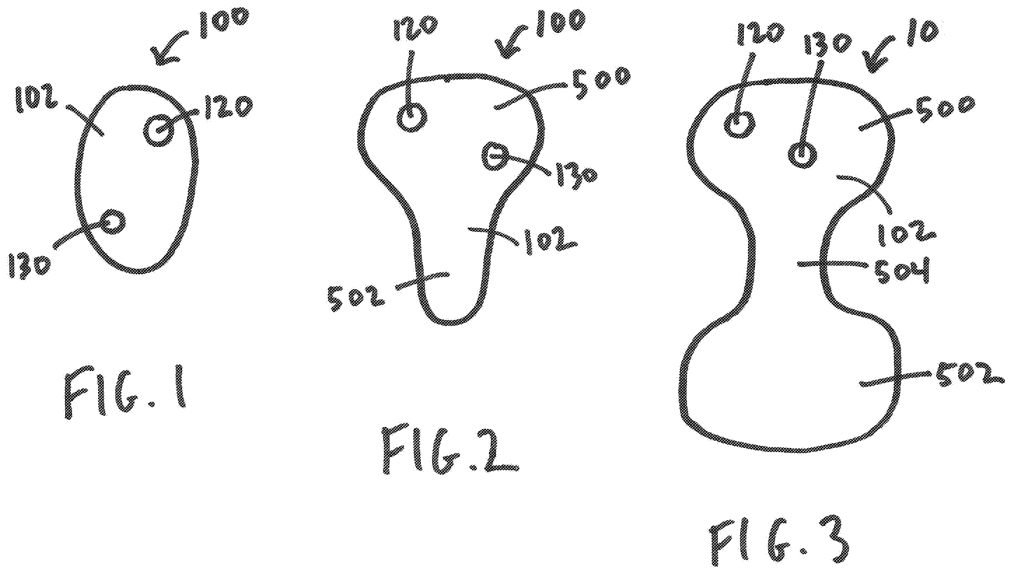

An exemplary device of the present disclosure is shown in FIG. 1. As shown therein, an exemplary device 100 of the present disclosure is relatively small and configured for comfortable placement into the anus, such as being at or about 2 mm to 5 mm in diameter, or perhaps smaller or larger. Said device 100 may comprise one or both of a bioimpedance sensor or element 120 and/or a pressure sensor 130, operable using a power source 160 (such as a battery, which may be rechargeable) so to power said sensors or elements 120, 130. Bioimpedance sensors or elements 120 of the present disclosure are configured to obtain conductance data/information of/from anorectal tissue as well as to obtain conductance data/information of/from fecal matter, noting that fecal matter can have 3-4 times the conductance of rectal tissue. Bioimpedance sensors or elements 120, or other one or more additional elements 132, of the present disclosure can also be configured to detect intestinal gas, which has a relatively low conductance, noting that if only intestinal gas is detected by bioimpedance sensors or elements 120, the person using said device 100 may, in some situations, choose to flatulate (fart), without any sort of liquids being expelled. Bioimpedance sensors or elements 120 of the present disclosure are therefore configured to determine a conductance difference between rectal tissue and fecal matter. Pressure sensors 130 of the present disclosure are configured to determine at least an increase or change in pressure from an initial pressure level, such as an initial pressure level with device 100 is positioned in the rectal cavity without fecal matter present, and an increase or change in pressure when fecal matter becomes present, contacting device 100, and signaling upcoming fecal expulsion.

As such, conductance data/information from bioimpedance sensors or elements 120 and/or pressure data/information from pressure sensor(s) 130 can be obtained within the body, and said data/information can be transmitted wirelessly (radio frequency, Bluetooth, etc.), via a wireless transmitter 140, to an external device 250, such as a smartphone, smartwatch, or other portable device, so to alert a user of device 100, or a caregiver of said user of device 100, that defecation may be soon or imminent due to changes in conductance and/or pressure, with said changes due the presence of fecal matter adjacent to device 100 within the rectal cavity. Users may suffer from rectal hyposensitivity, not being able to sense in a normal way the contents in the rectum. In at least one embodiment, the signals of involuntary defection are transmitted to an iPhone or an iPhone-like device (an external device 250) that alerts the person of the involuntary event to go to the toilet.

FIG. 1 shows an exemplary device 100 of the present disclosure, comprising an outer shell 102 and further comprising at least one bioimpedance sensor or element 120 and at least one pressure sensor 130 thereon/therein. Such a device is shown as being ovular, but other shapes configured for insertion into the rectal cavity, such as round (or having one or more rounded portions), are disclosed herein, so to allow for ease of insertion into the rectal cavity and expulsion during defecation. FIG. 2 shows another embodiment of an exemplary device 100 of the present disclosure, having a first bulbous/enlarged portion 500 and a smaller portion 502, whereby the first bulbous/enlarged portion 500 is relatively larger (such as a larger volume, larger diameter portion, etc.) than smaller portion 502, so that first bulbous/enlarged portion 500 can fit within the rectal cavity and so that smaller portion 502 can fit within the anus. FIG. 3 shows yet another embodiment of an exemplary device 100 of the present disclosure, having a first bulbous/enlarged portion 500, a smaller portion 502, and a second bulbous/enlarged portion 504, whereby the first bulbous/enlarged portion 500 and the second bulbous/enlarged portion 504 are relatively larger (such as a larger volume, larger diameter portion, etc.) than smaller portion 502, so that first bulbous/enlarged portion 500 can fit within the rectal cavity, so that smaller portion 502 can fit within the anus, and so that the second bulbous/enlarged portion 504 can be present outside of the anus, such as to provide a user of device 100 with a portion of the device 100 to physically contact to facilitate re-insertion or to hold the device 100 in place, as may be necessary.

Procedurally, an exemplary device 100 to the present disclosure can be inserted into the rectum/anus, and when operated, the device can obtain impedance/conductance and/or pressure data, and transmit the same to an external device 250. If the impedance/conductance data and/or pressure data changes to a level indicative of the presence of feces, consistent with the description referenced herein, data can be transmitted to the external device 250 to alert the user of device 100 that the user needs to go to the restroom to defecate (so to avoid involuntary defecation). As such, devices 100 of the present disclosure are configured to obtain impedance/conductance and/or pressure data over time, and send data, information, and/or a signal to an external device 250 that can be used to alert the user for the need to go to the restroom.

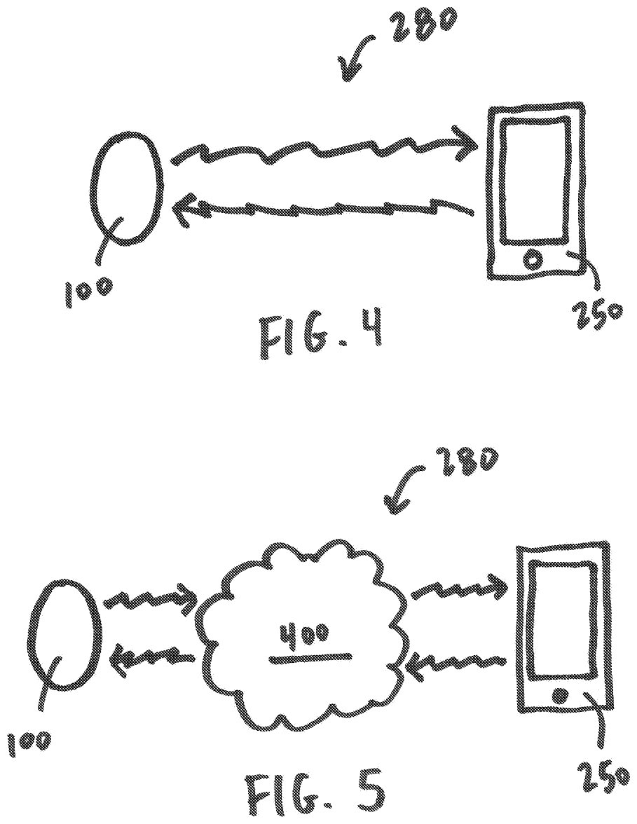

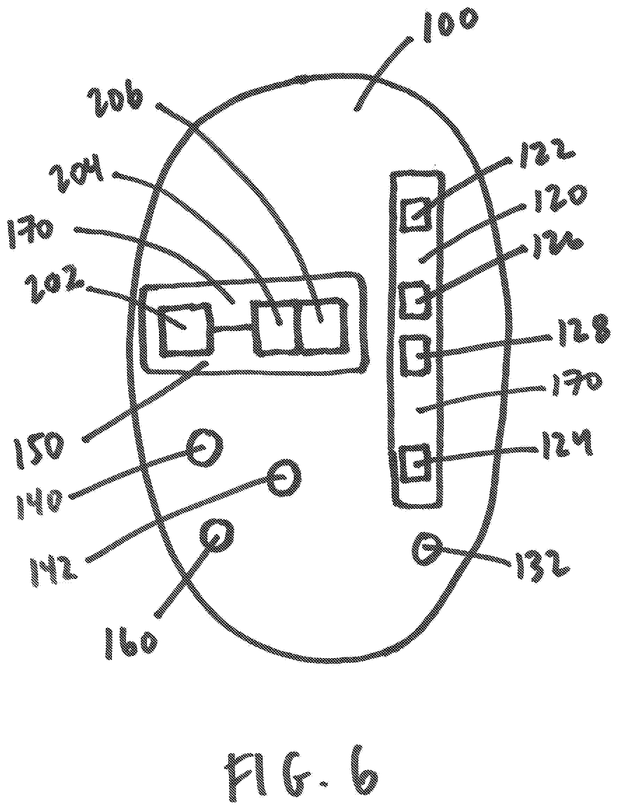

FIG. 4 depicts an exemplary system 280 of the present disclosure, comprising a device 100 and an external device 250, whereby the device 100 and the external device 250 can communicate in one direction (such as from device 100 to external device 250) and optionally in a reverse direction (such as from external device 250 to device 100). Communication from device 100 to external device 250 can be made to, for example, send a signal from device 100 to external device 250 to alert the user of device 100/system 280 to go to the restroom as defecation is imminent. Communication from external device 250 to device 100 can be made to, for example, reset device 100 (so to clear some current data from said device 100), etc. FIG. 5 depicts an exemplary system 280 of the present disclosure, whereby device 100 and external device 250 can communicate unidirectionally or bidirectionally through a network 400 instead of directly, as shown in FIG. 4.

Exemplary devices 100 useful with the present disclosure are also configured so to obtain impedance data and/or pressure data within the rectal cavity and/or anus, such as by way of sensors or elements 130 configured to detect intestinal gas. To accomplish the same, exemplary devices 100 useful with the present disclosure may comprise various electrical components, sensors, and the like, as described in further detail herein. For example, and exemplary device 100 useful with the present disclosure may comprise an impedance element 120, as shown in FIG. 6, configured to obtain impedance data within the gastrointestinal tract. Such an impedance element 120 and referenced herein, for example, may comprise several electrodes, such as a first excitation electrode 122 and a second excitation electrode 124 configured to generate an electric field that is detectable using a first detection electrode 126 and a second detection electrode 128, for example. Detection electrodes 126, 128 could be physically positioned in between excitation electrodes 122, 124 for example, so to obtain impedance data as desired.

Exemplary devices 100 useful with the present disclosure may contain various elements in lieu of or in addition to impedance element 120 noted above, such as, for example, a pressure sensor 130 configured to obtain pressure data within the rectal cavity and/or anus, and optionally one or more additional elements 132 which may comprise, for example, a camera with a light source configured to obtain visual data within the rectal cavity and/or anus, a pH sensor configured to obtain pH data within the rectal cavity and/or anus, a radiopaque marker configured to be detected using a scanner configured to detect a radiopaque marker within the body, an electromagnetic marker configured to be detected using a scanner configured to detect an electromagnetic marker within the body, a mucosa-attached electromagnetic stimulator (through non-invasive external stimulation), a gyroscopic sensor (a gyroscope) configured to obtain gyroscopic data, including but not limited to gyroscope-based angle data with the digestive tract, and/or a strain gauge and chemosensors such as gas sensors (exemplary sensors 120) embedded therein. Said data obtained using impedance element 120, pressure sensor 130, and/or one or more additional elements 132 can be transmitted from device 100 from within the body, for example, to an external device 250 outside of the body, such as a smartphone, a tablet, a laptop or desktop computer, a wired telephone, a non-smartphone cellular telephone, etc., by way of a wireless transmitter 140 within said device 100. Device 100 may also receive instructions from external device 250, such as instructions to operate or cease operation of one or more of impedance element 120, pressure sensor 130, and/or one or more additional elements 132, using a wireless receiver 142 within said device 100. In various embodiments, wireless transmitter 140 could also receive instructions/data, so said transmitter 140 could be considered as a transmitter/receiver.

In at least one embodiment, one or more additional elements 132 may comprise a printed circuit, such as a printed circuit board (PCB), which may be/comprise a substrate 170 as referenced in further detail herein, configured to measure electrical/electromyography (EMG) activity (exemplary data) within the rectal cavity and/or anus, for example. Said data can, in various embodiments, be obtained in addition to various mechanical measurements, such as pressure data obtained by pressure sensor 130 as referenced herein, impedance data obtained using impedance element(s) 120, and potential gyroscope-accelerometer-based angles obtained using one or more additional elements 132.

In at least one embodiment of a device 100 of with the present disclosure, said device 100 is configured to deliver electrical current to stimulate motility of the colon, such as to, for example, induce defecation for individuals with constipation, and the like. In such an embodiment, impedance elements 120 (such as electrodes 122, 124), or one or more additional elements 132 configured as an electrical stimulating element, can deliver an electric current, powered by power source 160, to stimulate motililty of the colon.

A computer element 150, as referenced in further detail herein, could also be positioned within device 100, and connected (via wires, traces, and/or wirelessly) to one or more of impedance element 120 (including, for example, electrodes 122, 124, 126, and/or 128), pressure sensor 130, one or more additional elements 132, wireless transmitter 140, and/or wireless receiver 142, so to, for example, control operation of said items. Computer element 150 could direct operation of said items without input from external device 250, and/or could direct operation of said items with input from external device 250, such as instructions from external device 250 received by wireless receiver 142 and transmitted to computer element 150.

Impedance element 120 (including, for example, electrodes 122, 124, 126, and/or 128), pressure sensor 130, one or more additional elements 132, wireless transmitter 140, wireless receiver 142, and/or computer element 150 could be powered using a power source 160, such as a battery. Power source 160 could be a single-use battery or a rechargeable battery, as may be desired. Alternative the energy supply can come from body-powered sources and elements.

Impedance element 120 (including, for example, electrodes 122, 124, 126, and/or 128), pressure sensor 130, one or more additional elements 132, wireless transmitter 140, wireless receiver 142, computer element 150, and/or power source 160 could be connected to one or more substrates 170, such as shown in FIG. 6. Such a substrate 170 could comprise plastic and/or metal, for example.

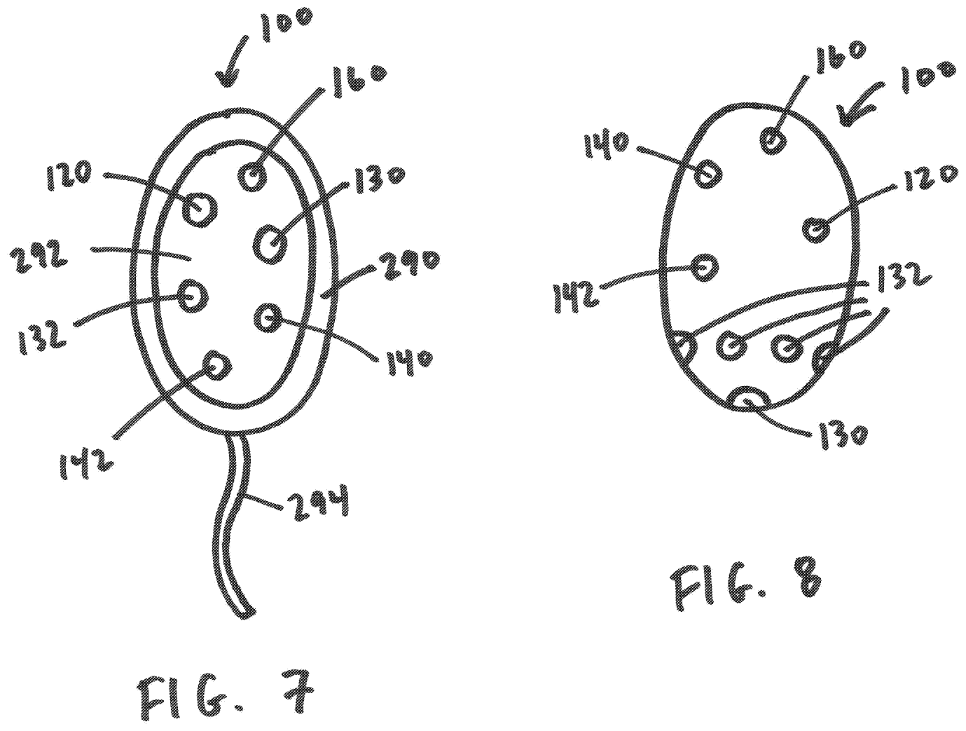

FIG. 7 shows an embodiment of a device 100 of the present disclosure, comprising (at least in part) an external capsule 290 encapsulating a spongy portion 292. External capsule 290 may comprise any number of materials that may generally dissolve in the presence of a liquid, such as gelatin or other materials, and spongy portions 292 may comprise any number of materials configured to absorb/receive liquids. A threaded element 294 may be connected to spongy portion 292, and spongy portion 292 may have any number of impedance elements 120, pressure sensors 130, or additional elements 132 therein or thereon, such as to obtain impedance/conductance data, pressure data, and/or other data, consistent with the present disclosure. Procedurally, a user may insert such a device into the anal cavity or rectum, and the external capsule 290 will ultimately dissolve at least in part, allowing liquids from the anal cavity or rectum to enter the spongy portion 292. Threaded element 294 can then be at least partially present outside the body to facilitate removal of device 100 as referenced herein. Conductance/impedance changes, pressure changes, etc., may then be detected by device 100 based on the presence of said liquids within the spongy portion 292, and a signal sent to an external device 250, such as referenced herein, based on said liquid presence, to alert the user to remove the device 100 (such as by pulling threaded element 294), potentially defecate, and/or potentially replace the device 100 with another, fresh/new device 100. Various elements, such as, for example, wireless transmitters 140, wireless receivers 142, power sources 160, impedance elements 120, and/or one or more additional elements configured as electrical stimulating elements, configured to stimulate portions of the colon 316, may be used in this and other device 100 embodiments.

An additional device 100 embodiment of the present disclosure is shown in FIG. 8. As shown therein, device 100 may comprise at least one pressure sensor 130, and one or more additional elements 132 configured as an electrical stimulating elements positioned along a relative outside of device 100. Other elements, such as, for example, wireless transmitters 140, wireless receivers 142, power sources 160, etc., may also be used. Pressure sensor 130, such as a distally positioned pressure sensor 130 positioned at a relative end of the rectum 312 or within the anal cavity 314, can detect pressure, and if there is relatively low anal pressure or spontaneous anal relaxation, for example, the electrical stimulating elements (elements 132) can be activated so to, for example, stimulate rectal/anal muscle contraction (directly or through a neural pathway) to facilitate defecation (such as to avoid constipation, for example).

While various embodiments of devices, systems, and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A device, comprising:
an outer shell configured for placement into the anus; and
at least one of a bioimpedance sensor or a pressure sensor positioned within and/or upon the outer shell.

2. The device of claim 1, wherein the at least one of the bioimpedance sensor or pressure sensor is configured to obtain conductance data/information of/from anorectal tissue as well as to obtain conductance data/information of/from fecal matter.

3. The device of claim 1, wherein the device is ovular in shape.

4. The device of claim 1, further comprising:
a first enlarged portion and a smaller portion, whereby the first enlarged portion is relatively larger than the smaller portion.

5. The device of claim 4, further comprising:
a second enlarged portion whereby the second enlarged portion is relatively larger than the smaller portion.

6. The device of claim 1, further comprising:
an external device, whereby said device and said external device can communicate bidirectionally.

7. The device of claim 1, further comprising:
a first excitation electrode, a second excitation electrode, a first detection electrode and a second detection electrode positioned within and/or upon the outer shell of the device

8. The device of claim 7, wherein the first detection electrode and the second detection electrode are physically positioned between the first excitation electrode and the second excitation electrode.

9. The device of claim 1, further comprising:
at least one additional element selected from the group consisting of a camera, a light source, a pH sensor, a radiopaque marker, an electromagnet marker, a mucosa attached electromagnetic stimulator, a gyroscopic sensor, a strain gauge, a chemosensor, a wireless transmitter, a wireless receiver, and a printed circuit board.

10. The device of claim 1, wherein the at least one of the bioimpedance sensor or the pressure sensor is configured to determine a change in pressure within an anal cavity when the device is positioned therein, and wherein the device further comprises:
one or more electrical stimulating elements positioned along a relative outside of the device.

11. A device, comprising:
an external capsule encapsulating a spongy portion; and
a plurality of sensors or elements in communication with the spongy portion, the plurality of sensors or elements configured to obtain data based on the presence of liquid in the spongy portion.

12. The device of claim 11, wherein the external capsule comprises a material that dissolves in the presence of the liquid, and the spongy portion comprises a material configured to absorb or receive the liquid.

13. The device of claim 11, further comprising:
a threaded element connected to the spongy portion.

14. The device of claim 11, wherein one or more sensors or elements of the plurality of sensors or elements are configured to obtain impedance/conductance data and pressure data relative to the device when the device is positioned in an anal cavity.

15. A method of using a device, comprising the steps of:
inserting a device into an anus of a patient, the device configured to obtain conductance data/information of/from anorectal tissue as well as to obtain conductance data/information of/from fecal matter present within the anus;
collecting a first set of pressure and bioimpedance data, wherein the first set of data is indicative of the absence of feces;
collecting a second set of pressure and bioimpedance data wherein the second set of data is indicative of the presence of feces; and
transmitting the first set of pressure and bioimpedance data and the second set of pressure and bioimpedance data to an external device.

16. The method of claim 15, wherein the second set of pressure and bioimpedance data comprises a pressure reading higher than a pressure reading of the first set of pressure and bioimpedance data.

17. The method of claim 15, wherein the second set of pressure and bioimpedance data comprises a impedance reading lower than an impedance reading of the first set of pressure and impedance data.

18. The method of claim 15, wherein said device comprises an external capsule encapsulating a spongy portion, and wherein the step of collecting the second set of pressure and bioimpedance data is performed after the external capsule is at least partially dissolved and when environmental liquids have entered the spongy portion.

19. The method of claim 18, wherein the device comprises a threaded element connected to the spongy portion, and wherein the method further comprises the step of:
removing the device from the anus of the patient by pulling the threaded element.

20. The method of claim 15, wherein said device comprises electrical stimulating elements positioned along a relative outside of the embodiment, and wherein the method further comprises the step of:
activating the electrical stimulating elements when the first set of pressure and bioimpedance data or second set of pressure and bioimpedance data indicates a low pressure.
